(19) United States
(12) Patent Application Publication
(10) Pub. No.: US 2010/0080846 A1
(43) Pub. Date: Apr. 1, 2010

(54) DIPYRIDAMOLE AND ACETYLSALICYLIC ACID FORMULATIONS AND PROCESS FOR PREPARING SAME

(76) Inventors: Avshalom Ben-Menachem, Kokav Yair (IL); Ilan Zalit, Rosh Ha Ayin (IL)

Correspondence Address: KENYON & KENYON LLP, ONE BROADWAY, NEW YORK, NY, 10004, US

(21) Appl. No.: 12/567,688

(22) Filed: Sep. 25, 2009

Related U.S. Application Data
(60) Provisional application No. 61/194,473, filed on Sep. 25, 2008.
(60) Provisional application No. 61/203,258, filed on Dec. 18, 2008.

(51) Int. Cl. A61K 9/48; A61K 9/52; A61P 9/10

(52) U.S. Cl. 424/452; 514/161; 424/451; 424/463; 424/457

(57) ABSTRACT

The present invention provides pharmaceutical formulations of dipyridamole and acetylsalicylic acid, methods of making thereof, and methods of using thereof.

### RELATED APPLICATION

This application claims the benefit of Provisional Application Ser. No. 61/194,473, filed Sep. 25, 2008, and Provisional Application Ser. No. 61/203,258, filed Dec. 18, 2008. The content of both applications are incorporated herein in their entirety by reference.

### FIELD OF THE INVENTION

The invention relates to pharmaceutical formulations of dipyridamole in pellet form and of acetylsalicylic acid in pellet form, and methods of making the same.

### BACKGROUND OF THE INVENTION

Dipyridamole (2,6-bis-(diethanolamino)-4,8-dipiperidino-(5,4-d)-pyrimidine) is reported to be clinically used as an active substance with antithrombotic and antiaggregatory activity.

An extended release form of administration of dipyridamole would apparently have the advantage of facilitating a reduction in the number of administrations per day, which would supposedly lead to better patient compliance. This is of significant importance with regard to long-term medication. Another advantage is that delayed absorption would reportedly lead to more uniform levels in the blood, thus avoiding or minimizing blood level peaks, which can apparently cause deleterious side effects, and avoiding sub-therapeutic levels of active substance, that can occur with instantaneous forms during longer dosage intervals. In other words, safety, compatibility, and effectiveness of the preparation can be increased. Due to the obvious advantages of an extended release form of dipyridamole, there have been previous efforts to develop such a form.

However, the physical and biochemical properties of dipyridamole would seem to suggest that it is completely unsuitable for the typical development of an extended release form. Apparently, the biological half-life of dipyridamole is relatively short-existing levels in the blood reportedly drop quickly, and a uniform dipyridamole level in the blood can apparently be obtained only if the active substance is constantly absorbed. Dipyridamole is reportedly soluble only in aqueous medium in the acidic range; more specifically, apparently above a pH of 4 the substance is practically insoluble in water. This seems to mean that dipyridamole can be dissolved only in the upper gastro-intestinal tract and consequently absorbed, whereas at the higher pH values occurring in the intestinal region it remains insoluble and is not absorbed.

It is known that acetylsalicylic acid, being an inhibitory substance, counteracts the aggregation of human blood platelets. It has been reported that acetylsalicylic acid inhibits the enzyme cyclooxygenase in the blood platelets and thus inhibits the biosynthesis of the aggregation-promoting thromboxane A₂. Apparently, as the dosage increases, the antithrombotic activity of acetylsalicylic acid increases, but also at the same time its inhibitory effect on the cyclooxygenase of the blood vessel walls is increased, which probably indirectly has a negative influence on the synthesis of the aggregation-inhibiting substance prostacyclin. It has been suggested that the lowest possible dose of acetylsalicylic acid be used. See, Lancet, III (1979) 1213, Prostaglandins and Medicine 4 (1980) 439. On the other hand, it has been recommended that higher doses be used since, as the dosage increases, the antithrombotic activity of acetylsalicylic acid is increased even if the biosynthesis of prostacyclin and thromboxane is already inhibited. See, Prostaglandins, Leukotrienes and Medicine 12 (1983), 235.

Since it has been reported that no stable uniform formulation can be obtained from acetylsalicylic acid in direct combination with dipyridamole, French Patent No. 2,368,272 proposes spatial separation of the components from one another in layered tablets or jacket/core tablets. In particular, one option is that the dipyridamole can be contained in the outer layer and the acetylsalicylic acid can be contained in the inner layer of the tablet. Other combination possibilities include formulations in which either the dipyridamole is contained in the inner layer and surrounded by a layer which is soluble in intestinal juice and the acetylsalicylic acid is surrounded by a layer which is soluble in gastric juice, or both the dipyridamole and the acetylsalicylic acid are surrounded by a layer which is soluble in gastric juice. The proposed weight ratio between acetylsalicylic acid and dipyridamole is from 4:1 to 1:4.

U.S. Pat. No. 4,694,024 purportedly discloses successive administration of dipyridamole followed by a delayed release of O-acetyl-salicylic acid.

U.S. Pat. No. 4,367,217 (“the '217 patent”) purportedly discloses compositions in which dipyridamole and carboxylic acid are combined together into spheroid particles which are surrounded with a dialysis membrane that consists essentially of acid-insoluble lacquers soluble in intestinal juices.

U.S. Pat. No. 6,015,577 (“the '577 patent”) purportedly discloses a combination of dipyridamole and/or mopidamol and acetylsalicylic acid which releases the two components simultaneously in the gastrointestinal tract. The '577 patent is the U.S. patent listed in the U.S. Food and Drug Administration's Approved Drug Products with Therapeutic Equivalence Evaluations (“the Orange Book”) for Aggrenox®. The '577 patent states that it is well known that “acetylsalicylic acid is not free from traces of acetic acid which are formed by cleavage of the acetylsalicylic acid during storage. The free acetic acid reacts with the dipyridamole to form hygroscopic salts and dipyridamole-acetic acid esters which cause the dipyridamole to spoil.” The '577 patent further states that, to prevent the spoiling processes one or both of the components should have a separating layer. The examples given by the '577 patent include dipyridamole in the form of pellets or granules with a coating which is insoluble in gastric juices but soluble in intestinal juices and/or the acetylsalicylic acid cores or tablets with a coating which is resistant to acetic acid and is very quickly dissolved in the gastric juices; dipyridamole granulate, separating granulate, and acetylsalicylic acid granulate compressed into three layer tablets; dipyridamole granulate packed into a capsule together with a coated tablet provided with a protective coating, or a film coated tablet, containing the acetylsalicylic acid; and compressed tablets of acetylsalicylic acid with a film coating combined with the dipyridamole pellets that can be also coated.

Copending U.S. patent application Ser. No. 11/353,498, published as U.S. Patent Application Publication No. US20070184110, discloses extended release formulations of dipyridamole formed in a tablet solid form having a diameter of about 1.5 mm and about 3 mm, and an immediate release formulation of acetylsalicylic acid, wherein the extended release and the immediate release formulations are combined in a capsule.

There is no convenient formulation developed for pediatric and elder populations which can be given as a sprinkle capsule. In addition, formulations having a number of active substances risk interaction between them resulting in formulations which are unstable or contain degradants, often limiting the activity of the active ingredients. Therefore, there is a need for stable formulation and for formulation that can be used by pediatric and elder populations.

One aspect of the invention is directed to developing delayed release dipyridamole formulations that can withstand the acidity of the stomach and release in the more basic environment of the intestines.

### SUMMARY OF THE INVENTION

In one embodiment, the present invention encompasses a pharmaceutical formulation comprising:

(i) pellets comprising dipyridamole, and

(ii) pellets comprising acetylsalicylic acid,

wherein components (i) and (ii) are physically separated.

Preferably, at least one of components (i) and (ii) is encapsulated in an capsule. Preferably, the pellets comprising acetylsalicylic acid are encapsulated in an inner capsule. Preferably, the pellets comprising dipyridamole are encapsulated in an outer capsule. Preferably, the inner capsule comprising the pellets comprising acetylsalicylic acid is encapsulated in the outer capsule. Preferably, the pellets comprising dipyridamole are in extended release form. Preferably, the pellets comprising acetylsalicylic acid are in immediate release form.

The invention relates to a capsule containing pellets of dipyridamole and pellets of acetylsalicylic acid, wherein dipyridamole pellets differ in their release profile in the gastrointestinal tract from acetylsalicylic acid pellets. The invention further provides a process for the manufacture of capsule formulations. The process is especially suitable for the manufacture of the dipyridamole-acetyl salicylic acid capsules of the present invention.

### BRIEF DESCRIPTION OF THE DRAWINGS

FIG. 1 shows the effect of coating thickness on the dissolution profiles of dipyridamole in coated dipyridamole pellets.

FIG. 2 shows the dissolution profiles of dipyridamole in combination dipyridamole/acetylsalicylic acid capsules of the present invention and in acetylsalicylic acid/extended release dipyridamole 25 mg/200 mg capsules sold under the trademark Aggrenox®.

FIG. 3 shows the dissolution profiles of acetylsalicylic acid in combination dipyridamole/acetylsalicylic acid capsules of the present invention and in Aggrenox®.

FIG. 4 shows the effect of pellet size on the dissolution profiles of acetylsalicylic acid.

### DETAILED DESCRIPTION OF THE INVENTION

As used herein, the term “pellet” refers to particles that are non-compressed, for example, a granule or a sphere. The term “pellet” does not encompass compressed tablets.

As used herein, unless otherwise defined, the term “particle size of smaller than A” refers to particle diameter as determined by particle sieving technique, specifically, at least 80% of the particles pass through a sieve with size A openings; the term “particle size of bigger than B” refers to particle diameter as determined by particle sieving technique, specifically, at least 80% of the particles retain on a sieve with size B openings. Examples of commonly-used sieves are sieves characterized by mesh numbers. The mesh number is a measure of how many openings there are per linear inch of a screen.

As used herein, the term “stable” in the context of a dipyridamole/acetylsalicylic acid pharmaceutical formulation refers to a formulation in which each of dipyridamole's degradants, whether the chemical structure is known or not, has a weight that is not more than 0.2% based on the initial weight of the dipyridamole in the formulation, and in which each of acetylsalicylic acid's degradants, including salicylic acid and any unknown degradants, has a weight that is not more than 3.0% (if the degradant is known) and not more than 0.2% (if the degradant is unknown) based on the initial weight of acetylsalicylic acid, after storage at 25° C. and 60% relative humidity for 2 years or under any other accelerated conditions that simulate storage at 25° C. and 60% relative humidity for 2 years (e.g., 3 months at 40° C. and 75% relative humidity; 6 months at 30° C. and 60% relative humidity).

Extended release layers are layers that extend or “slow down” the release of coated substance, such as dipyridamole and/or organic acid. The extended release layers may comprises:
1. polymers that resist dissolving in the gastric juices of the stomach and are more soluble at the pH normally found in the intestine. These polymers are often referred to as enteric (coating) polymers, as they are usually used to prevent dissolution of the active material before reaching the intestine; and
2. polymers that have pH-independent solubility/swellability.

In one embodiment, the present invention encompasses a pharmaceutical formulation comprising pellets comprising dipyridamole and pellets comprising acetylsalicylic acid, wherein the pellets comprising dipyridamole are physically separated from the pellets comprising acetylsalicylic acid.

Preferably, the pellets comprising dipyridamole are in extended release form. The pellets comprising dipyridamole may be in a form of a matrix or may be coated with an extended release layer. Preferably, the pellets comprising dipyridamole are coated with an extended release layer.

Preferably, the pellets comprising acetylsalicylic acid are in immediate release form. The pellets comprising acetylsalicylic acid may be uncoated or coated with an immediate release layer. Preferably, the pellets comprising acetylsalicylic acid are uncoated.

Preferably, the pellets comprising dipyridamole and the pellets comprising acetylsalicylic acid are encapsulated, preferably in a capsule.

In a preferred embodiment, the present invention encompasses a capsule comprising (i) pellets comprising dipyridamole, and (ii) pellets comprising acetylsalicylic acid. The pellets comprising acetylsalicylic acid may be uncoated or coated with an immediate release layer. Preferably, the pellets comprising dipyridamole are coated with an extended release layer. Preferably, the pellets comprising acetylsalicylic acid are uncoated.

In one embodiment, the present invention encompasses a pharmaceutical formulation comprising pellets comprising dipyridamole and pellets comprising acetylsalicylic acid, wherein the pellets comprising dipyridamole and the pellets comprising acetylsalicylic acid are kept separate by encapsulation. Preferably, the formulation is in the form of a capsule comprising another capsule.

Preferably, the pellets comprising dipyridamole are in extended release form. More preferably, the pellets comprising dipyridamole are coated with an extended release layer.

Preferably, the pellets comprising acetylsalicylic acid are in immediate release form. The pellets comprising acetylsalicylic acid may be uncoated or coated with an immediate release layer. Preferably, the pellets comprising acetylsalicylic acid are uncoated.

In one embodiment, the pellets comprising acetylsalicylic acid may be incorporated into an inner capsule, wherein the inner capsule is inserted into a larger outer capsule. The outer capsule also encapsulates the pellets comprising dipyridamole. The pellets comprising dipyridamole may be uncoated, or preferably coated with an extended release layer. The pellets comprising acetylsalicylic acid may be coated with an immediate release layer, or preferably uncoated.

In a preferred embodiment, the present invention encompasses a pharmaceutical formulation comprising an outer capsule that encapsulates:

(i) pellets comprising dipyridamole; and
(ii) an inner capsule that encapsulates pellets comprising acetylsalicylic acid.

Preferably, each of the pellets comprising dipyridamole comprises:

(a) an inner core comprising a granulate comprising at least one organic acid, and

(c) a drug layer comprising dipyridamole.

Preferably, each of the pellets comprising dipyridamole further comprises (b) an enteric coating layer on top of the inner core. Preferably, the drug layer comprising dipyridamole is on top of the enteric coating. Preferably, each of the pellets comprising dipyridamole further comprises (d) an extended release layer on top of the drug layer comprising dipyridamole.

Preferably, at least one of the granulate comprising at least one organic acid, the enteric coating layer, the drug layer comprising dipyridamole, the extended release layer, and the pellets comprising acetylsalicylic acid further comprises at least one pharmaceutically acceptable excipient. Preferably, the at least one pharmaceutically acceptable excipient in the enteric coating layer or in the extended release layer is a plasticizer.

Preferably, the outer capsule has a sufficient interior volume to house both the inner capsule and the desired amount of the pellets comprising dipyridamole. Preferably, the inner capsule is size 5. Preferably, the outer capsule is size 000, 00, or 0 elongated.

In a preferred embodiment, a size 00 elongated capsule is filled with pellets comprising dipyridamole and a size 5 capsule pre-filled with pellets comprising acetylsalicylic acid.

In any embodiment of the present invention, the inner and outer capsules may independently be hard or soft gelatin capsules or any pharmaceutically acceptable capsule known in the art.

Preferably, the weight ratio of the active agent encapsulated in the outer capsule to the active agent in the inner capsule is between about 15 and about 0.1, preferably between about 10 and about 0.5, more preferably between about 8 and about 1. For example, in one embodiment, the dipyridamole in the outer capsule is present in an amount of about 200 mg and the acetylsalicylic acid in the inner capsule is present in an amount of about 25 mg.

Preferably, the amount of dipyridamole is between about 10% and about 75%, preferably about 20% to about 60%, more preferably about 30% to about 50%, and most preferably about 35% to about 45% by weight of the final pellet comprising dipyridamole.

In any embodiment of the present invention, the pellet comprising dipyridamole may comprise an organic acid, preferably a carboxylic acid. Preferably, the organic acid used in the formulations of the invention is hydrophilic and capable of maintaining a pH at which the dipyridamole is soluble. Preferably, the organic acid is capable of maintaining a pH of about 4 or less. Example of suitable hydrophilic acids include, but are not limited to, those having at least one hydroxyl group and/or carbonyl group. Preferably, the organic acid has a pKa of about 5 or less, preferably 4.5 or less, and more preferably about 4.2 or less. Preferred carboxylic acids include, but are not limited to, fumaric, tartaric, citric, succinic, adipic, and malic acid.

Preferably, the organic acid is present in an amount sufficient to maintain a pH capable of dissolving the dipyridamole. Preferably, when a carboxylic acid is used in a pellet comprising dipyridamole, the ratio of the carboxylic acid to dipyridamole may be between about 1:10 and about 10:1 by weight, preferably between about 1:2 and about 2:1 by weight. Optionally, in an extended release formulation of any embodiment of the present invention, the molar ratio of the organic acid to dipyridamole is between about 1:2 and about 2:1.

Preferably, in a pellet comprising dipyridamole, the amount of organic acid is about 10% to about 70%, preferably about 20% to about 60%, more preferably about 30% to about 60%, and more preferably about 35% to about 50% by weight of the final pellet.

Preferably, in an inner core comprising at least one organic acid, the amount of organic acid is between about 10% to about 100%, preferably about 20% to about 90%, more preferably about 30% to about 85%, and more preferably about 40% to about 80% or about 50% to about 80% by weight of the inner core, and the remainder of the inner core is made up with at least one pharmaceutical excipient. The organic acid may be combined with the at least one pharmaceutically acceptable excipient, such as those described below. Preferably, the at least one pharmaceutically acceptable excipient is a diluent, preferably microcrystalline cellulose.

In any embodiment of the present invention, the pellets comprising dipyridamole pellets may be in the form of spheres. Each of the optionally spherical pellets preferably comprises an inner core, an optional enteric coating layer (first or inner layer) on top of the inner core, a drug layer (second layer) comprising dipyridamole on top of the inner core or the optional enteric coating layer, and an optional extended release layer (third or outer layer) on top of the drug layer.

The inner core may comprise an organic acid, preferably a carboxylic acid, more preferably tartaric acid, and optionally at least one pharmaceutically acceptable excipient. Preferably, the inner core comprises a granulate comprising at least one organic acid and at least one pharmaceutically acceptable excipient. Preferably, the at least one pharmaceutically acceptable excipient is microcrystalline cellulose.

The drug layer comprises dipyridamole and preferably further comprises at least one pharmaceutically acceptable excipient. Preferably, the drug layer comprises between about 10% and about 100%, preferably between about 30% and about 95%, more preferably between about 50% and about 90%, and most preferably between about 70% and about 85% dipyridamole by weight and the remainder of the drug layer is made up by at least one pharmaceutical excipient.

Preferably, the at least one pharmaceutically acceptable excipient in the drug layer is a binder, a lubricant, or a combination thereof. Preferably, the binder is hydroxypropyl cellulose, polyvinylpyrrolidone, or copovidone. Preferably, the lubricant is sodium lauryl sulfate, magnesium stearate, or stearic acid. Preferably, hydroxypropyl cellulose and sodium lauryl sulfate are present in the drug layer.

The drug layer may be present in an amount of about 10% to about 80%, preferably about 20% to about 70%, more preferably about 30% to about 50%, and more preferably about 35% to about 45%, by weight of the final pellets comprising dipyridamole.

Preferably, in any embodiment of the present invention, each of the pellets comprising dipyridamole have an extended release layer. Preferably, the extended release layer is applied on top of the drug layer. The extended release layer may be present in an amount of about 0.5% to about 20%, preferably about 1% to about 18%, more preferably about 1% to about 15%, and more preferably about 1% to about 10%, by weight of the final pellets comprising dipyridamole. Preferably, the amount of the extended release layer is about 1.5% to about 7%, more preferably about 2% to about 6%, by weight of the final pellet.

Preferably, in any embodiment of the present invention, each of the pellets comprising dipyridamole include an enteric coating. The enteric coating is preferably applied to the inner core of the dipyridamole pellets. The enteric coating may be present in an amount of about 1% to about 15%, preferably about 2% to about 10%, more preferably about 3% to about 9%, and more preferably about 3% to about 8%, by weight of the final pellets.

Preferably, in the formulations of the invention, at least one enteric polymer is used to form an enteric coating layer (first layer) on the inner core comprising at least one organic acid. Preferably, a pH-independent extended release polymer is used for coating the drug layer comprising dipyridamole (second layer) to form the extended release layer (third layer).

The extended release layer and/or the enteric coating layer allow(s) a certain amount of the active material to be dissolved in the stomach and continued to be released at a defined rate on reaching the intestine. This is preferably achieved by controlling the amount of organic acid released form the core at different pHs so that in acidic conditions the organic acid is not released from inner core, while at increased pH the organic acid is to be released from core, and consequently increased the solubility of the dipyridamole in the drug layer. Optionally, the coating is, for example, “thin” enough to allow a defined amount of drug to be released from the pellets even at the acid conditions of the stomach. Preferably, the extended release layer does not comprise an enteric polymer. Optionally, an extended release layer can comprise one or more enteric polymers.

In any embodiment of the present invention, the extended release layer may comprise at least one extended release polymer that include, but are not limited to, known extended release polymers and hydrophobic agents used in pharmaceutical formulations, for example, ethylcellulose polymers (for example, those sold under the tradename Ethocel®), hydroxypropyl methylcellulose polymers, and polyvinylpyrrolidine polymers. Preferred extended release polymers include, but not limited to, hydroxypropyl methylcellulose polymers.

Preferably, the extended release layer comprises about 50% and about 100%, preferably about 60% to about 95%, more preferably about 65% to about 85%, and more preferably about 70% to about 80% by weight extended release polymer(s) and the remainder of the extended release layer is made up with at least one pharmaceutical excipient.

In any embodiment of the present invention, the enteric coatings layer may comprise at least one enteric polymer. Suitable enteric polymers include, but are not limited to, known enteric polymers, for example, methacrylic polymers. Preferred enteric polymers including, but not limited to, methacrylic acid-ethyl acrylate copolymer, methacrylic acid-methyl methacrylate copolymer (for example, Eudragit® S or L), hydroxypropyl methylcellulose phthalate, cellulose acetate phthalate, ethylcellulose phthalate, hydroxypropylmethylcellulose succinate, cellulose acetate succinate, hydroxypropylmethylcellulose hexahydrophthalate, cellulose acetate hexahydrophthalate, hydroxypropylmethylcellulose trimellitate, and combinations thereof.

Preferably, the enteric coating layer comprises about 50% to about 100%, preferably about 60% to about 95%, more preferably about 65% to about 85%, and more preferably about 70% to about 80% enteric polymer(s) by weight and the remainder of the enteric coating layer is made up with at least one pharmaceutical excipient.

Preferably, the extended release layer and/or the enteric coating layer comprise(s) at least one pharmaceutically acceptable excipient, preferably a plasticizer. Suitable plasticizers used in the formulation include, but are not limited to, acetyltributyl citrate, acetyltriethyl citrate, castor oil, diacetylated monoglycerides, dibutyl sebacate, diethyl phthalate, glycerin, polyethylene glycol, triacetin, tributyl citrate, and triethyl citrate. Preferably, the plasticizer is triethyl citrate.

Preferably, the plasticizer is present in an amount of about 15% to about 30%, preferably about 20% to about 27%, more preferably about 22% by weight of the extended release layer.

Preferably, the plasticizer is present in an amount of about 3% to about 20%, preferably about 6% to about 15%, more preferably about 8% by weight of the enteric coating layer.

Preferably, the plasticizer is present in an amount of about 5% to about 30% by weight of the pellet comprising dipyridamole. Preferably, the plasticizer is present in an amount of about 3% to about 20% by weight of the enteric coating layer and about 15% to about 30% by weight of the extended release layer.

Preferably, the pellets used in the formulation of the present invention each comprises:
an inner core comprising tartaric acid;
a first layer that is an enteric coating layer comprising methacrylic acid copolymer sold under the tradename Eudragit S-100;
a second layer that is a drug layer comprising dipyridamole; and
a third layer that is an extended release layer comprising a mixture of ethylcellulose and triethyl citrate.

Optionally, the extended release layer comprises a mixture of methacrylic acid copolymers (e.g. methacrylic acid copolymer, Type B NF, sold under the tradename Eudragit® S-100, and methacrylic acid copolymer, Type A NF, sold under the tradename Eudragit® L-100) and triethyl citrate. Preferably, the weight ratio of the methacrylic acid copolymer, Type B NF, methacrylic acid copolymer, Type A NF, and the triethyl acetate in the extended release layer is about 1:1:1.

Although dipyridamole is practically insoluble at a pH above 4, in vitro release tests with artificial intestinal juices having a pH of 5 to 7 have shown that dipyridamole may diffuse out of extended release formulation. The organic acid acidifies intestinal juices after penetrating into the dipyridamole-containing part of the extended release formulation. Despite the intestinal juices surrounding the extended release formulation, an acidic medium prevails within the extended release form. Thus, the dipyridamole may dissolved and diffuse out. And as the dipyridamole is absorbed, more dipyridamole is released continuously into the intestinal tract.

Preferably, the pellets comprising dipyridamole pellets for use in any embodiment of the present invention have a particle size smaller than about 2000 μm, more preferably smaller than about 1800 μm, smaller than about 1410 μm, smaller than about 1400 μm, smaller than about 1300 μm, and smaller than about 1200 μm. A particle size smaller than 1410 μm means that at least 80% of the particles passed through a US standard sieve size 14 (mesh number 12) that has openings in the size of 1410 μm.

The pellets comprising dipyridamole for use in any embodiment of the present invention can be prepared by any method known in the art that produces non-compressed formulations, including those disclosed in the '217 patent, the content of which is incorporated herein in its entirety by reference.

The pellet comprising dipyridamole can be in the form of a sphere. The pellets comprising dipyridamole may be produced by a process comprising: (1) preparing an inner core comprising an organic acid, preferably tartaric acid, and at least one pharmaceutically acceptable excipient, preferably a filler, more preferably microcrystalline cellulose, preferably using an extrusion and spheronization technique; (2) preferably applying the enteric coating layer on top of the inner core; (3) applying the drug layer on top of the inner core or the enteric coating layer; and (4) preferably applying an extended release layer on top of the drug layer.

In one embodiment, the inner core is prepared by an extrusion and spheronization technique comprising at least one of the following steps: (1) wet granulation of an organic acid and at least one pharmaceutically acceptable excipient for form a wet mass; (2) extruding the wet mass to form rod-shaped particles, preferably of uniform size; (3) spheronizing the rod-shaped particles to form spherical particles; (4) drying the spherical particles; and (5) separating the spherical particles of a desired particle size range by sieving. Preferably, the particle size is between about 840 μm and about 400 μm or between about 840 μm and about 420 μm, more preferably between about 840 μm and about 500 μm. A particle size range of between 840 μm and 420 μm can be achieved, for example, when at least 80% of the pellets pass through a US standard sieve size 20 (Tyler mesh number 20) that has openings in the size of 840 μm and at least 80% of the pellets retain on a US standard sieve size 40 (Tyler mesh number 35) that has openings in the size of 420 μm.

The enteric coating layer may be applied to the inner core by any conventional method. Preferably, the enteric coating layer is applied on top of the inner core using a coating solution comprising at least one enteric polymer and at least one pharmaceutical acceptable excipient, preferably a plasticizer. The coating process is preferably performed using a fluidized bed coater.

The drug layer comprising dipyridamole may be applied using any conventional method. Preferably, the drug layer comprising dipyridamole is applied on top of the inner core or the enteric coating layer using a hydro or hydro-alcoholic dispersion of dipyridamole and at least one pharmaceutical acceptable excipient, preferably a binder. The coating process is preferably performed using a fluidized bed coater.

The extended release layer may be applied to the drug layer by any conventional method. Preferably, the extended release layer is applied using a solution of at least one extended release polymer and at least one pharmaceutical acceptable excipient, preferably a plasticizer.

In any of the coating steps above, the coating processes may be performed using a fluidized bed coater. Preferably, the coater equipped with a bottom spray coating device (Wurster device). Preferably, the coater is a continuous fluidized bed such as those sold under the trademark Glatt®. The solution of dispersion used for coating is preferably an organic solution or dispersion of the coating materials. Preferably, the solution is an alcoholic solution.

The dipyridamole used in any embodiment of the present invention may be dipyridamole or any pharmaceutically acceptable form thereof. Suitable forms of dipyridamole include, but are not limited to, salts, solvates, anhydrates, hydrates, and polymorphs (including crystalline and amorphous forms).

The acetylsalicylic acid used in any embodiment of the present invention may be acetylsalicylic acid or any pharmaceutically acceptable form thereof. Suitable forms of acetylsalicylic acid include, but are not limited to, salts, solvates, anhydrates, hydrates, and polymorphs (including crystalline and amorphous forms).

Pharmaceutically acceptable salts of acetylsalicylic acid are those with pharmaceutically acceptable cations such as metal cations, ammonium, amine cations, and quaternary ammonium cations. Metal cations include, but are not limited to, alkali metals, such as lithium, sodium, and potassium, and alkaline earth metals, such as magnesium and calcium. It is also possible to use cationic forms of other metals, such as aluminum, zinc, and iron. pharmaceutically acceptable amine cations include, but are not limited to, those of primary, secondary, and tertiary amines. Amine cations include alkylamines, such as methyl-, dimethyl-, trimethyl-, ethyl-, dibutyl-, triisopropyl-, N-methylhexyl-, benzyl-, β-phenylethylamine, ethylenediamine, diethylenetriamine, piperidine, morpholine, piperazine, mono-, di- and triethanolamine, ethyldiethanolamine, and N-butylethanolamine. Other suitable amine salts include basic amine salts of lysine and arginine. Examples of suitable pharmaceutically acceptable quaternary ammonium cations include, but are not limited to, tetramethylammonium, tetraethylammonium, and benzyltrimethylammonium.

Preferably, the pellets comprising acetylsalicylic acid pellets for use in any embodiment of the present invention have between about 20% and about 70%, preferably between about 30% and about 50%, more preferably between about 35% and about 45% acetylsalicylic acid by weight.

In any embodiment of the present invention, the acetylsalicylic acid pellets may be coated with an immediate release coating. Such coatings include those that dissolve quickly to release the acetylsalicylic acid, including, but are not limited to, gastrosoluble compositions for film-coating of moisture sensitive solid particles such as a composition comprising hydroxypropylmethylcellulose (e.g. film coating system sold under the tradename SEPIFILM™ LP by SEPPIC Inc., Fairfield, N.J.), polyvinyl alcohol-polyethylene glycol graft copolymer (e.g. film coating systems sold under the tradenames Kollicoat® IR (by BASF Pharma Solutions) and SEPIFILM™ IR), and sugars.

Optionally, the immediate release coating is present in an amount of about 1% to about 20%, preferably about 1% to about 10%, and more preferably about 2% to about 5% by weight of the pellet comprising acetylsalicylic acid. Even more preferably, the immediate release coating is present in an amount of about 3% to about 4% by weight of the pellet comprising acetylsalicylic acid.

The pellets comprising acetylsalicylic acid of any embodiment of the present invention can be prepared by any method known in the art that produces non-compressed pellets.

In one embodiment, the pellets comprising acetylsalicylic acid pellet is prepared by an extrusion and spheronization technique comprising at least one of the following steps: (1) wet granulation of acetylsalicylic acid and at least one pharmaceutically acceptable excipient to form a wet mass; (2) extrusion of the wet mass to form rod-shaped particles, preferably of uniform size; (3) spheronizing the rod-shaped particles to form spherical particles; (4) drying the spherical particles; and (5) separating the particles of the desired particle size range by sieving. An immediate release coating may optionally be applied to the pellets.

The pellets comprising acetylsalicylic acid pellets for use in any embodiment of the present invention preferably have a particle size of between about 4000 μm and about 200 μm, more preferably between about 2500 μm and about 300 μm, and more preferably between about 1700 μm and about 420 μm. A particle size range of between 1000 μm and 590 μm can be achieved, for example, when at least 80% of the pellets pass through a US standard sieve size 18 (Tyler mesh number 16) that has openings in the size of 1000 μm and at least 80% of the pellets retain on a US standard sieve size 30 (Tyler mesh number 28) that has openings in the size of 590 μm.

If the pellets comprising acetylsalicylic acid are to be encapsulated in the inner capsule they preferably have a particle size between about 1000 μm and about 300 μm, more preferably between about 1000 μm and about 400 μm, more preferably between about 800 μm and about 600 μm.

A preferred pellet comprising acetylsalicylic acid comprises between about 20% and about 70%, preferably between about 30% and about 50%, and more preferably between about 35% and about 45% by weight acetylsalicylic acid. Preferably, the pellets comprising acetylsalicylic acid have a particle size of between about 1000 μm and about 200 μm, preferably between about 1000 μm and about 400 μm, and more preferably between about 800 μm and about 600 μm.

The pellets comprising acetylsalicylic acid defined above provide an improved combination of stability, dosage uniformity and dosage amount.

A skilled artisan, without due experimentation, can easily determine the methods, parameters, and equipment for mixing the ingredients or wet granulating the ingredients for producing the pellets comprising dipyridamole and the pellets comprising acetylsalicylic acid defined above. The skilled artisan will also be able to determine the conditions for performing the drying and milling steps using equipment commonly available in the art. As the skilled artisan knows, conditions such as amount of ingredients, temperature, and/or amount of wetness, among others, will affect the conditions necessary to carry out the process. For example, mixing of the organic acid and a pharmaceutically acceptable excipient can be performed using a high sheer mixer.

In any of the final formulations of any embodiment of the present invention, the weight ratio of dipyridamole to acetylsalicylic acid may preferably be between about 10 and about 0.5, more preferably between about 8 and about 1. For example, in one embodiment, the dipyridamole is present in 200 mg and the acetyl salicylic acid is present in 25 mg in the formulation.

Selection of excipients and the amounts to use can be readily determined by an experienced formulation scientist in view of standard procedures and reference works known in the art.

Excipients used in any formulation of the invention include, but are not limited to, binders, diluent, disintegrants, lubricants, glidants, extended release polymers (as described above), sweetening agent, coloring agent, flavoring agent, and plasticizer (as described above).

Suitable binders for the formulation include, but are not limited to, acacia, alginic acid, carbomer copolymer, carbomer interpolymer, copovidone, microcrystalline cellulose, dextrin, ethylcellulose, gelatin, glucose (liquid), guar gum, hydroxypropyl cellulose, maltose, methylcellulose, polyethylene oxide, polyvinylpyrrolidone, starch, and sodium carboxymethylcellulose. Preferred binders include hydroxypropyl cellulose, polyvinylpyrrolidone, and copovidone.

Preferably, in any embodiment of the present invention, the binder is present in an amount of about 1% to about 10%, more preferably about 2% to about 8%, for example, about 4%, by weight of the pellet comprising acetylsalicylic acid or the pellet comprising dipyridamole.

Preferred binders for the pellet comprising acetylsalicylic acid include hydrophilic polymers such as polyvinylpyrrolidone and copovidone. Preferably, the binder is hydroxypropyl cellulose (e.g., those sold under the tradename KLUCEL®), preferably present in an amount of about 1% to about 10%, more preferably about 3 to 6%, by weight of the pellet comprising acetylsalicylic acid.

Suitable lubricants which can optionally be used in the formulation include, but are not limited to, sodium lauryl sulfate, calcium stearate, glyceryl behenate, magnesium stearate, polyethylene glycol, sodium stearyl fumarate, stearic acid, talc, and zinc stearate. Preferred lubricants include sodium lauryl sulfate, magnesium stearate, and stearic acid. Preferably, in any embodiment of the present invention, the lubricant is present in an amount of about 1% to about 6%, more preferably about 1% to about 4% by weight of the pellet comprising acetylsalicylic acid or the pellet comprising dipyridamole. Preferably, for pellet comprising dipyridamole pellet, the lubricant is present in an amount of between about 3% and about 5% by weight of the pellet.

Suitable disintegrants which can optionally be used in the formulation include, but are not limited to, alginic acid, microcrystalline cellulose, croscarmellose sodium, crospovidone, maltose, polacrilin potassium, sodium starch glycolate, and starch. Preferably, the disintegrant is crospovidone. Preferably, in any embodiment of the present invention, the disintegrant is present in an amount of about 1% to about 4%, more preferably about 2% by weight of the pellet comprising acetylsalicylic acid or the pellet comprising dipyridamole.

Suitable diluent which can optionally used in the formulation include, but are not limited to, calcium carbonate, calcium phosphate (dibasic and/or tribasic), calcium sulfate, microcrystalline cellulose, dextrates, dextrin, dextrose excipient, fructose, kaolin, lactitol, lactose (anhydrous and/or monohydrate), maltose, mannitol, microcrystalline cellulose, sorbitol, starch, and sucrose. Preferably, the diluent is lactose or microcrystalline cellulose.

Preferably, in any embodiment of the present invention, the diluent is present in an amount of about 12% to about 60%, for example about 50% to about 60%, preferably about 55% by weight of the pellet comprising acetylsalicylic acid or the pellet comprising dipyridamole. Optionally, the diluent is present in an amount of about 36% by weight of the pellet comprising acetylsalicylic acid.

Suitable glidants which can optionally be used in the formulation include, but are not limited to, talc, kaolin, glycerol monostearate, silicic acid, magnesium stearate, and titanium dioxide.

Other suitable excipients which can optionally be used in the formulation include, but are not limited to, microcrystalline cellulose (e.g. those sold under the tradename AVICEL®), lactose, calcium phosphate, etc.

In any embodiment of the present invention, a preferred pharmaceutically acceptable excipient combination for the pellets comprising dipyridamole is microcrystalline cellulose (preferably between about 10% and about 15% by weight of the pellet), ethyl cellulose (preferably between about 2% and about 6% by weight of the pellet), triethyl citrate (preferably between about 0.5% and about 2% by weight of the pellet), hydroxypropyl cellulose (preferably between about 1% and about 5% by weight of the pellet), or sodium lauryl sulfate (preferably between about 2% and about 6% by weight of the pellet).

In any embodiment of the present invention, a preferred pharmaceutically acceptable excipient combination for the pellets comprising acetylsalicylic acid is microcrystalline cellulose (preferably between about 10% and about 20% by weight of the pellet), hydroxypropyl cellulose (preferably between about 2% and about 6% by weight of the pellet), or lactose (preferably between about 30% and about 50% by weight of the pellet).

A preferred embodiment of the present invention comprises an outer capsule that encapsulates:

(i) pellets, each of which comprising:
(a) an inner core comprising a granulate comprising at least one organic acid (preferably in an amount of between about 35% and about 45% by weight of the pellet) and microcrystalline cellulose (preferably in an amount of between about 10% and about 15% by weight of the pellet),
(b) an enteric coating on top of the inner core comprising at least one enteric polymer, preferably an anionic polymers with methacrylic acid as a functional group and dissolves only at a pH above 7.0 (e.g., those sold under the tradename EUDRAGIT® S 100).
(c) a drug layer on top of the enteric coating, comprising dipyridamole (preferably in an amount of between about 30% and about 40% by weight of the pellet), hydroxypropyl cellulose (preferably in an amount of between about 1% and about 5% by weight of the pellet), and sodium lauryl sulfate (preferably in an amount of between about 2% and about 6% by weight of the pellet), and
(d) an extended release layer comprising ethyl cellulose (preferably in an amount of between about 2% and about 6% by weight of the pellet) and triethyl citrate (preferably in an amount of between about 0.5% and about 2% by weight of the pellet); and

(ii) an inner capsule that encapsulates pellets comprising acetylsalicylic acid (preferably in an amount of between about 35% and about 45% by weight of the pellet), microcrystalline cellulose (preferably in an amount of between about 10% and about 20% by weight of the pellet), hydroxypropyl cellulose (preferably in an amount of between about 2% and about 6% by weight of the pellet), and lactose (preferably in an amount of between about 30% and about 50% by weight of the pellet).

Not to be limited by theory, but it is believed that the formulations of the present invention allow separation of the dipyridamole and acetylsalicylic acid formulations to promote dipyridamole and acetylsalicylic acid stability. Further, an extended release formulation may combine dipyridamole and at least one organic acid, such that the organic acid promotes the dissolution of the dipyridamole in the gastrointestinal environment.

In one embodiment, about 5% to about 30% by weight of the dipyridamole in the formulations of the present invention dissolves within about 60 minutes, after being mixed at 37° C. in a dissolution medium of 900 ml of 0.1N HCl in a USP Apparatus I (basket), under a mixing speed of 100 rpm, in a dissolution medium of 900 ml 0.1N HCl. Particularly preferred dissolution profiles are as follows:

Suggested dissolution profile of dipyridamole:

| Time | % dissolved |
|---|---|
| 60 min | 5-30 |
| 120 min | 30-60 |
| 180 min | 50-70 |
| 360 min | 60-90 |
| 700 min | 80-100 |

In one embodiment, about 60% to about 90% by weight acetylsalicylic acid in the formulations of the present invention dissolves within 20 minutes, after being mixed in a USP Apparatus I (basket), under a mixing speed of 100 rpm at 37° C. in a dissolution medium of 900 ml 0.1N HCl. Particularly preferred dissolution profiles are as follows:

Suggested dissolution profile of acetylsalicylic acid:

| Time | % dissolved |
|---|---|
| 10 min | 40-70 |
| 20 min | 60-90 |
| 30 min | 80-100 |

The above preferred dissolution profile can be achieved by:

1. For pellets comprising dipiridamole: by varying the amount of the extended release layer applied to the pellets comprising dipyridamole, by varying the extended release layer excipient(s), by changing other parameters such as the amount of acid (e.g. tartaric acid), thickness, and/or type of enteric polymer on the acid core (e.g. Eudragit® S 100), particle size of dipiridamole, pellets size, and any other process parameters.
2. For pellets comprising acetylsalicylic acid: by varying the percent of acetylsalicylic acid is the pellets, pellets size, the type and amount of filler and binder, and any other process parameters.

Dissolution studies of the capsules of the present invention demonstrated that the dipyridamole in the form of coated pellets with a none-pH dependent diffusion layer exhibit a similar dissolution file to the commercially available dipyridamole pellets sold by Boehringer Ingelheim under the trademark Aggrenox®/Asasantin® (Product website: http://www.aggrenox-asasantin.com/com/Home/index.jsp; U.S. Food and Drug Administration Application No. (NDA) 020884). According to its label (available at http://www.accessdata.fda.gov/drugsatfda_docs/label/2007/020884s012lbl.pdf and http://www.aggrenox.com), each Aggrenox® hard gelatin capsule contains 200 mg dipyridamole in an extended-release form and 25 mg aspirin as an immediate-release sugar-coated tablet. In addition, each capsule contains the following inactive ingredients: acacia, aluminum stearate, colloidal silicon dioxide, corn starch, dimethicone, hypromellose, hypromellose phthalate, lactose monohydrate, methacrylic acid copolymer, microcrystalline cellulose, povidone, stearic acid, sucrose, talc, tartaric acid, titanium dioxide and triacetin. Each capsule shell contains gelatin, red iron oxide and yellow iron oxide, titanium dioxide and water.

For example, capsules filled with the pellets comprising dipyridamole and pellets comprising acetylsalicylic acid of the present invention were tested for dissolution of dipyridamole. The method used a USP Apparatus I, with a mixing speed of 100 rpm and at a temperature of 37° C., in a first medium (medium 1,900 ml 0.1N HCl) for 1 hour, with samples of dissolution medium taken at 1 hour and analyzed; and a second medium (medium II, 900 ml buffer, preferably NaH₂PO₄ buffer, having a pH of 5.5) for 1 to 12 hours. The pellets comprising dipyridamole in the dipyridamole/acetylsalicylic acid capsules of the present invention have a similar dissolution profile to the dipyridamole pellets in Aggrenox®.

In one embodiment, the pellets comprising dipyridamole have 3% by weight of extended release layer, and the weight percentages of dipyridamole dissolved are 25% in medium I after 1 hour and 96% in medium II after 12 hours.

In one embodiment, the pellets comprising acetylsalicylic acid comprises 40% acetylsalicylic acid by weight and have a size of between 1000 μm and 590 μm as measured by sieving, and made by extruder with a mesh size of about 800 μm, and the weight percentages of acetylsalicylic acid dissolved are above 50% in medium I after 30 minutes and above 85% in medium I after 60 minutes.

In one embodiment, the pellets comprising acetylsalicylic acid comprises 40% acetylsalicylic acid by weight and have a size of between 1680 μm and 840 μm as measured by sieving, and made by extruder with a mesh size of about 1200 μm, and the weight percentages of acetylsalicylic acid dissolved are above 40% in medium I after 30 minutes and above 70% in medium I after 60 minutes.

The capsule within a capsule embodiment of the present invention is surprisingly stable, particularly when the inner capsule comprises acetylsalicylic acid and is encapsulated within an outer capsule comprising dipyridamole. Under the same conditions of moisture, light, etc, the capsule encapsulating both dipyridamole and an inner capsule encapsulating acetylsalicylic acid usually has better stability properties than Aggrenox® (see Table 10). This is advantageous as when using Aggrenox® it is advised not to leave the bottle open because atmospheric conditions lead to degradation. The capsule within a capsule embodiment of the present invention is resistant to such atmospheric degradation. This is particularly advantageous for elderly and pediatric patients.

The formulations of the present invention may be used to reduce the risk of stroke in patients who have had a stroke or a transient ischemic attack.

The formulations of the present invention may be administered as “sprinkle” pellets. For example, the pellets comprising dipyridamole and the pellets comprising acetylsalicylic acid can be sprinkled on the patient's food and to be taken orally by the patient. Optionally, the capsules encapsulating the pellets can be broken open to sprinkle the pellets.

The capsule formulations of the present invention may be prepared by any encapsulation processes known in the state of the art. With respect to the capsule within a capsule embodiment, the inner capsule is filled prior to insertion into the outer capsule.

In a preferred embodiment of the present invention, the outer capsule is filled with at least one inner capsule, preferably one inner capsule, and another solid dosage form, preferably pellets. Preferably, the at least one inner capsule is placed in the capsule shell body prior to the filling with the other solid dosage form. The inner capsule is filled prior to being placed in the capsule shell body. This may be done by any methods known in the art or the method of the present invention. Preferably the inner capsule is capsule size 5 and the outer capsule is size 000, 00, or 00 elongated.

The process of the present invention may be used to produce any of the formulations of the present invention discussed above.

In a preferred embodiment, a capsule is filled with an inner capsule and pellets. Preferably, the inner capsule contains pellets comprising acetylsalicylic acid and the outer capsule contains said inner capsule and pellets comprising dipyridamole. More preferably, the pellets comprising dipyridamole are extended-release formulations and the pellets comprising acetylsalicylic acid are immediate release formulations.

In a preferred embodiment, a capsule is filled with pellets comprising different active agents, preferably pellets comprising acetylsalicylic acid and pellets comprising dipyridamole. Preferably, the pellets comprising dipyridamole are extended-release formulations and the pellets comprising acetylsalicylic acid are immediate release formulations. Preferably, the dipyridamole pellets are each coated with an extended release layer.

Having described the invention with reference to certain preferred embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

### EXAMPLES

### Example 1

### Preparation of the Inner Cores of Pellets Comprising Dipyridamole

TABLE 1
List of ingredients in the inner core (tartaric acid pellet)

| | | Weight |
|---|---|---|
| Raw Material Description | Weight (g) | (mg/capsule) |
| PART I | | |
| Tartaric acid NF | 3900.0 | 220.00 |
| Microcrystalline cellulose | 1300.0 | 73.00 |
| (sold under the tradename | | |
| AVICEL ® PH 101) | | |
| Wetting Liquid | | |
| Purified Water USP | 400.0 | |
| Purified Water USP | 400.0 | |
| Purified Water USP | 200.0 | |
| Theoretical end weight | 5200.0 | 293.00 |

The tartaric acid, followed by the AVICEL®, were inserted into a Diosna P10 mixer-granulator and mixed for 2 minutes. 400 ml of purified water was added and mixed for 100 seconds. A further 400 ml of purified water was added and mixed for another 100 seconds. Another 200 ml of purified water was added and mixed for 50 seconds. The resulting granulate was discharged and passed through an Nica™ E-140 extruder with a 0.6 mm screen and an impeller speed of 50 rpm. The extrudants were charged to a spheronizer (Spheronizer 700, Caleva Process Solutions Ltd., UK) for 7 minutes at 500-800 rpm. Drying of the resultant pellets was carried out in a fluid bed spray dryer (Glatt model GPCG 5) with an inlet air temperature of 60° C. and an outlet air temperature of not more than 50° C. The resultant spheres were sieved through a 20 mesh (0.841 mm) sieve. The spheres that were retained on the sieve were disregarded. The remaining spheres were sieved through a 35 mesh (0.500 mm) sieve. The spheres that were retained on the sieve were discarded.

### Example 2

### Preparation of the Enteric Coating Layer of Pellets Comprising Dipyridamole

TABLE 2
List of ingredients in the enteric coating layer

| | Weight | Weight |
|---|---|---|
| Raw Material Description | (g) | (mg/capsule) |
| Tartaric acid pellets from Example 1 | 4500.0 | 293.00 |

Enteric coating layer

| | | |
|---|---|---|
| PART I | | |
| Eudragit ® S100 (anionic polymer of | 226.5 | 14.70 |
| methacrylic acid and methacrylates) | | |
| Ethanol, 95% | 1941.0 | |
| PART II | | |
| Triethyl citrate | 25.8 | 1.70 |
| Talc, USP extra fine | 51.6 | 3.40 |
| Ethanol 95% | 974.0 | |
| Theoretical end weight (pellets) | 4804.0 | 312.80 |

1. The Eudragit® S100 was added to 95% ethanol and mixed for about 1.5 hours until a clear solution was obtained.

2. The talc was added to 95% ethanol and mixed in Silverson mixer (mesh 60) for 20 minutes, and the triethyl citrate was added while stirring.

3. The mixtures from step 1 and step 2 were combined in the Silverson mixer (mesh 60) for 5 minutes, and transferred to a fluid bed spray dryer (Glatt model GPCG 5).

4. The pellets obtained in Example 1 were coated by top spraying with the mixture from step 3 under the following conditions: inlet temperature 35° C., outlet temperature of not more than 30° C., nozzle: 1.2 mm, atomizing air pressure of 2.5 bar, air flow of 450 m³/hr.

5. The coated pellets were dried form 30 minutes with an outlet temperature of not more than 30° C.

6. The coated pellets were sieved through a US size 18 mesh (with size 1.00 mm opening) sieve. The spheres that were retained on the sieve were discarded.

### Example 3

### Preparation of the Drug Layer of Pellets Comprising Dipyridamole

TABLE 3
List of ingredients in the drug layer

| | | Weight |
|---|---|---|
| Raw Material Description | Weight (g) | (mg/capsule) |
| Coated tartaric acid pellets from Example 2 | 3500.0 | 312.80 |

Drug layer

| | | |
|---|---|---|
| hydroxypropylcellulose LF (Klucel ® LF) | 179.0 | 16.00 |
| Sodium lauryl sulfate (SLS) | 269.0 | 24.00 |
| Dipyridamole, powder | 2238.0 | 200.00 |
| Deionized water | 7484 | |
| Theoretical end weight (coated pellets) | 6186.0 | 552.80 |
| *Qt of solution to be sprayed | 10045.0 | |
| Qt of solids to remain on core | 2686.0 | |
| % coating | 76.7 | |
| Solution Concentration | 26.7% | |

The Klucel® LF was added to the water and stirred for an hour until a clear solution was obtained. The sodium lauryl sulfate was added and stirred for a further 15 minutes. The dipyridamole was added under stirring until the dipyridamole powder was blended in. The dipyridamole solution was passed through a microfluidizer GLUT 5 with 400 and 200 micron holes and kept stirred at all times. The solution was transferred to a fluid bed spray dryer (Glatt model GPCG 5) equipped with a Wurster device. The spheres obtained in Example 2 were coated with the dipyridamole solution under the following conditions: inlet temperature 50° C., outlet temperature of not more than 30° C., nozzle: 1.2 mm, atomizing air pressure of 1.5 bar (150000 Pascal), air flow of 270 m³/hr (0.075 m³/s). The coated spheres were sieved through a US size 18 mesh (with size 1.00 mm opening) sieve. The spheres that were retained on the sieve were discarded.

### Example 4

### Preparation of the Extended Release Layer of Pellets Comprising Dipyridamole

TABLE 4
List of ingredients in the extended release layer

| | | Weight |
|---|---|---|
| Raw Material Description | Weight (g) | (mg/capsule) |
| Drug pellets from Example 3 | 2500.0 | 552.80 |

Extended release layer

| | | |
|---|---|---|
| PART I | | |
| Ethylcellulose (Ethocel ®) | 117.0 | 25.80 |
| Triethyl citrate | 31.6 | 7.00 |
| Ethanol, 95% | 1515.0 | |
| Theoretical end weight (coated pellets) | 2648.6 | 585.60 |
| *Qt of solution to be sprayed | 1670.0 | |
| Qt of solids to remain on core | 148.6 | |
| % coating | 5.9 | |
| Solution Concentration | 8.9% | |

The Ethocel® was added to the 95% ethanol and stirred for an hour until a clear solution was obtained. The triethyl citrate was added and stirred for a further 1 hour. The solution was transferred to a fluid bed spray dryer (Glatt model GPCG 5) equipped with a Wurster device. The spheres produced in Example 3 were coated with the solution under the following conditions: inlet temperature 35° C., outlet temperature of not more than 30° C., nozzle: 1.2 mm, atomizing air pressure of 2.5 bar (250000 Pascal), air flow of 450 m³/hr (0.125 m³/s). The coated spheres were sieved through a US size 16 mesh (with size 1.19 mm opening) sieve. The spheres that were retained on the sieve were disregarded.

### Example 5

### Preparation of Pellets Comprising Acetylsalicylic Acid

TABLE 5
List of ingredients in the pellets comprising acetylsalicylic acid

| | Weight | Weight |
|---|---|---|
| Raw Material Description | (g) | (mg/capsule) |
| PART I | | |
| Acetylsalicylic acid | 1250.0 | 25.00 |
| Microcrystalline cellulose (Avicel ® PH 102) | 500.0 | 10.00 |
| Hydroxypropylcellulose (Klucel ®) | 150.0 | 3.00 |
| Lactose, anhydrous | 1250.0 | 25.00 |
| Wetting liquid | | |
| Purified Water USP | 800.0 | |
| Theoretical end weight | 3150.0 | 63.00 |

The acetylsalicylic acid, followed by the Avicel® PH 102, followed by the Klucel®, followed by the anhydrous lactose were inserted into a Diosna P10 mixer-granulator and mixed for 2 minutes. 800 ml of purified water was added using a peristaltic pump at 50 rpm over 350 seconds. The granulate was discharged. The wet mass was converted into wet spheres using extruder (with extruder mesh sizes of 0.8 mm or 1.2 mm) and spheronizer technology. The wet spheres were further dried using fluid bed drier to form dry pellets.

The dry pellet were sieved as follows:

For 800 μm diameter pellets (extruder mesh sizes of 0.8 mm): The pellets were sieved through 1000 μm net and then through a 590 μm net. The retained on the 590 μm net were above 85% (of the total original pellets wt.).

For 1200 μm diameter pellets (extruder mesh sizes of 1.2 mm): The pellets were sieved through 1680 μm net and then through a 840 μm net. Above 85% of the pellets (by weight of the total pellets sieved) was retained on the 840 μm net.

### Example 6

### Encapsulation

The pellets comprising dipyridamole were prepared according to Examples 1 to 4. The pellets comprising acetylsalicylic acid were prepared as recited in Example 5.

The pellets comprising acetylsalicylic acid from Example 5 were encapsulated in inner capsules (size No. 5). The inner capsules were them inserted into outer capsules (size No. 00 elongated). The pellets comprising dipyridamole were from Example 4 were then added to the outer capsules with the inner capsules, and the outer capsules were closed.

### Example 7

### Dissolution of Dipyridamole in Pellets Comprising Dipyridamole with Different Amounts of Ethylcellulose (Ethocel®)

### Testing the Effect of Ethylcellulose (an External, Water Insoluble Polymer Sold Under the Tradename Ethocel®) Coating on the Dissolution of Dipyridamole

Pellets comprising dipyridamole were prepared according to the methods described in Examples 1 to 4, but with ethylcellulose (Ethocel®) coatings of various thicknesses. The relative thicknesses is expressed as the weight percentage of the ethylcellulose coating based on the total weight of the pellets. Pellets with coatings of 1.7%, 2.6%, 3.1%, and 3.5% by weight of the final pellets weight were prepared.

### Dissolution Test Conditions

### Stage 1 (Relevant to Both Dipyridamole and Acetylsalicylic Acid)

A USP Apparatus I (basket) with a mixing speed of 100 rpm at 37° C., for 1 hour in a dissolution medium of 900 ml 0.1N HCl (medium I) was used. Samples of the dissolution medium were taken over the course of the 1 hour and analyzed.

### Stage 2 (Relevant Only to Dipyridamole)

The medium I describe above is pumped out from the vessel followed by addition of 900 ml of NaH₂PO₄ buffer having a pH of 5.5 (medium II) for 1 to 12 hours, when a mixing speed of 100 rpm at 37° C. was used. Samples of the dissolution medium were taken over the course of the 2 to 12 hours and analyzed.

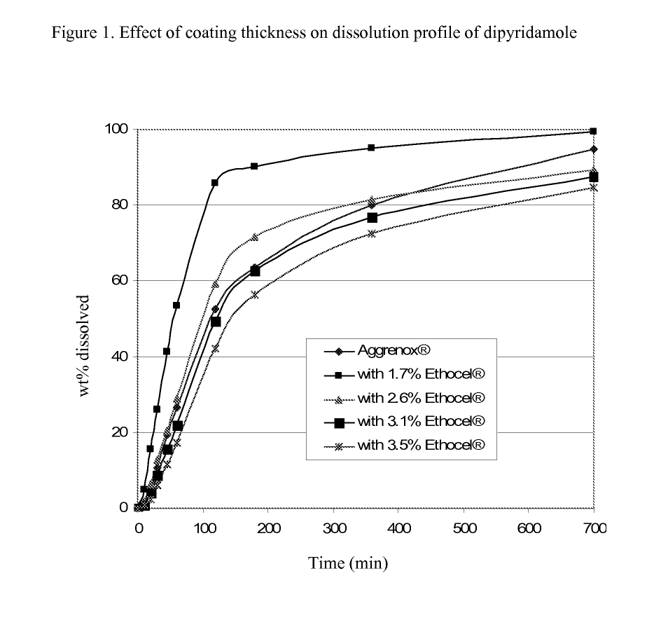

The results are listed in Table 6 and the dissolution profiles are shown in FIG. 1.

TABLE 6
Dissolution of dipyridamole in pellets comprising dipyridamole

| | | Formulation of the present invention with different amount of Ethocel ® | | | |
|---|---|---|---|---|---|
| | Aggrenox ® | 1.7% Ethocel ® | 2.6% Ethocel ® | 3.1% Ethocel ® | 3.5% Ethocel ® |
| min | wt % dissolved | wt % dissolved | wt % dissolved | wt % dissolved | wt % dissolved |
| 0 | 0.0 | 0.0 | 0.0 | 0.0 | 0.0 |
| 10 | 1.5 | 4.8 | 1.7 | 1.0 | 0.8 |
| 20 | 5.0 | 15.5 | 6.4 | 4.1 | 2.3 |
| 30 | 10.6 | 26.1 | 12.7 | 8.7 | 6.2 |
| 45 | 19.5 | 41.4 | 20.6 | 15.7 | 11.5 |
| 60 | 26.7 | 53.6 | 28.9 | 21.9 | 17.3 |
| 120 | 52.7 | 85.8 | 59.2 | 49.4 | 42.3 |
| 180 | 63.5 | 90.3 | 71.8 | 62.7 | 56.3 |
| 360 | 80.1 | 95.2 | 81.6 | 77.0 | 72.5 |
| 700 | 94.9 | 99.3 | 89.2 | 87.7 | 84.7 |

### Example 8

### Dissolution Profiles of Dipyridamole and Acetylsalicylic Acid in Aggrenox® in Comparison to Those of Dipyridamole and Acetylsalicylic Acid in Formulation of the Present Invention

Capsules were prepared according to Example 6 using inner capsule filled with pellets comprising acetylsalicylic acid pellets prepared according to Example 5 and pellets comprising dipyridamole pellets prepared according to Examples 1-4. The weight percent of ethylcellulose (Ethocel®) coating is 3.5% based on the total weight of the pellets comprising dipyridamole pellets.

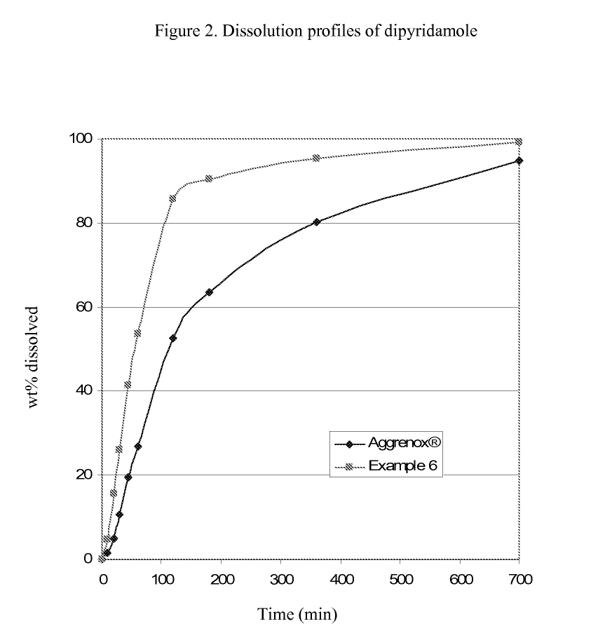

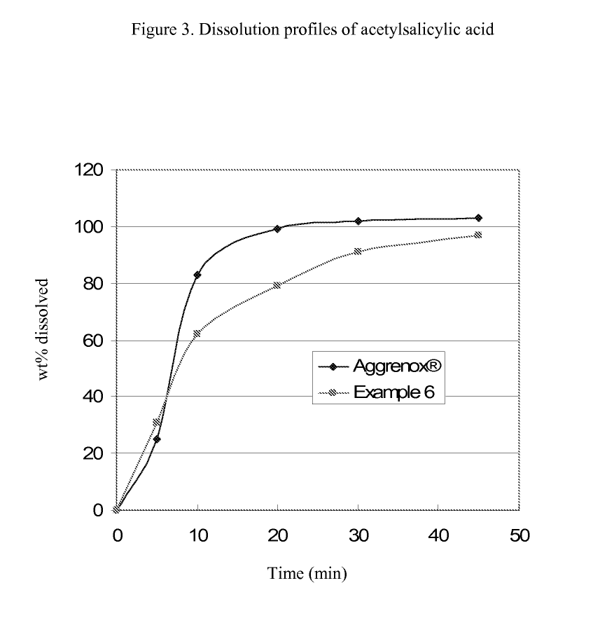

The dissolution test was conducted under the condition described in Example 7 above. The results are listed in Tables 7 and 8 and the dissolution profiles are shown in FIGS. 2 and 3.

TABLE 7
Dissolution of dipyridamole
Dipyridamole wt % dissolved vs. time

| | | Formulation of |
|---|---|---|
| | | present invention |
| | Aggrenox ® | (Example 6) |
| min | wt % dissolved | wt % dissolved |
| 0 | 0.0 | 0.0 |
| 10 | 1.5 | 4.8 |
| 20 | 5.0 | 15.5 |
| 30 | 10.6 | 26.1 |
| 45 | 19.5 | 41.4 |
| 60 | 26.7 | 53.6 |
| 120 | 52.7 | 85.8 |
| 180 | 63.5 | 90.3 |
| 360 | 80.1 | 95.2 |
| 700 | 94.9 | 99.3 |

TABLE 8
Dissolution of acetylsalicylic acid
Acetylsalicylic acid wt % dissolved vs time

| | | Formulation |
|---|---|---|
| | | of invention |
| | Aggrenox ® | (Example 6) |
| min | wt % dissolved | wt % dissolved |
| 0 | 25 | 31 |
| 10 | 83 | 62 |
| 20 | 99 | 79 |
| 30 | 102 | 91 |
| 45 | 103 | 97 |

### Example 9

### Dissolution of Acetylsalicylic Acid in Pellets of Different Sizes

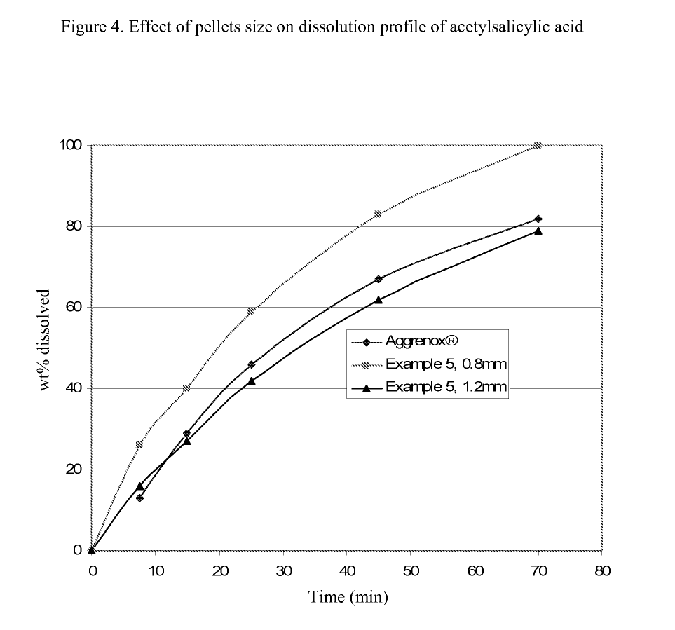

Pellets comprising acetylsalicylic acid were prepared according to Example 5 but with different extruder mesh sizes (0.8 mm and 1.2 mm). The dissolution test was conducted under the stage 1 condition described in Example 7 above. The results are listed in Table 9 and the dissolution profiles are shown in FIG. 4.

TABLE 9
Dissolution of acetylsalicylic acid

| | Acetylsalicylic acid wt % dissolved vs. time | | |
|---|---|---|---|
| | | Formulation of invention | |
| | | pellets made with | pellets made with |
| | | extruder mesh size | extruder mesh size |
| | Aggrenox ® | 0.8 mm | 1.2 mm |
| min | wt % dissolved | wt % dissolved | wt % dissolved |
| 0 | | 0 | 0 |
| 7.5 | 13 | 26 | 16 |
| 15 | 29 | 40 | 27 |
| 25 | 46 | 59 | 42 |
| 45 | 67 | 83 | 62 |
| 70 | 82 | 100 | 79 |

### Example 10

### Stability of Dipyridamole and Acetylsalicylic Acid in Aggrenox® in Comparison to Those of Dipyridamole and Acetylsalicylic Acid in Formulation of the Present Invention

Aggrenox® is supplied in a box that has a hygroscopic material in the lid (package cover) to minimize degradation by moisture. To evaluate the stability of the dipyridamole and acetylsalicylic acid under situations where the package is not sealed, a set of conditions for comparison between the formulation of the present invention (Example 6) and an Aggrenox® sample were carried out and analyzed by HPLC. The results are presented in Table 10.

### HPLC Conditions:

Column: Luna 5μ Phenyl-Hexyl, 250×4.6 mm 5μ

Column temp.: room temperature

Flow: 1.2-1.5 mL/min (depends on HPLC pressure condition)

Injection volume: 10 μL

Injection wash: water:acetonitrile, 1:1 vol.

Detector: 230 nm, 10 mm cell path length

Mobile phase:

Solution A: 0.01M phosphate buffer pH 2.0*

Solution B: acetonitrile:formic acid, 99:1 vol. *Buffer preparation: dissolve 1.4 g NaH₂PO₄.H₂O(S-13a) in 1 L water, titrate to pH=2.0 with phosphoric acid.

Gradient:

| Time (min) | % Solution A | % Solution B |
|---|---|---|
| 0 | 80 | 20 |
| 8 | 50 | 50 |
| 15 | 50 | 50 |
| 25 | 80 | 20 |

The results presented shows a clear advantage of the formulation of the invention, in terms of dipyridamole stability. According to the specification of International Conference on Harmonisation (ICH), there should be not more than 0.2% by weight unknown impurity. After stability testing at accelerated conditions (simulating storage at room temperature for 2 years), the main degradant of dipyridamole in Aggrenox® was very close to the maximum limit according to the ICH specification, whereas the main degradant for the formulation of present invention tested under the aforementioned conditions was present at a level much lower than the ICH specification. For acetylsalicylic acid tested under the same conditions, the known main degradant, salicylic acid, was within, but far from, the maximum limit allowed by the ICH specification of 3.0% for both Aggrenox® and the formulation of the present invention.

TABLE 9
Stability test results

| | | | | | Acetylsalicylic acid | |
|---|---|---|---|---|---|---|
| | | | | | stability data | |
| | Dipyridamole stability data | | | | | Acetylsalicylic |
| | | | Dipyridamole pellets in | | | acid tablet in |
| | | | Aggrenox ® | | | Aggrenox ® |
| | Example 6 | | (lot no. 603619) | | | (lot no. |
| | Degradant | | Degradant | | Example 6 | 603619 |
| | with the | Total | with the | Total | Total | Total |
| Storage | highest | concentration | highest | concentration | concentration | concentration of |
| condition | concentration | of degradants | concentration | of degradants | of degradants | degradants |
| t = 0 | | ≦0.05 | | 0.26 | 0.32 | 0.67 |
| 55° C., 3 days | 0.07 | 0.07 | 0.35 | 0.64 | 4.11 | 3.88 |
| container - close | | | | | | |
| 55° C., 1 day | 0.1 | 0.13 | — | — | 3.74 | — |
| container - open | | | | | | |
| 40° C., 1 day | 0.08 | 0.08 | 0.27 | 0.62 | 4.02 | 3.94 |
| container - open | | | | | | |
| 40° C., 2 days | 0.06 | 0.06 | 0.27 | 0.57 | — | 3.88 |
| container - open | | | | | | |
| ~30° C., 1 week | 0.07 | 0.07 | 0.2 | 0.47 | — | 5.43 |
| container - open | | | | | | |
| 40° C./75% RH, | | ≦0.05 | | | 0.32 | 0.67 |
| 3 months | | | | | | |
| container - close | | | | | | |
| 40° C./75% RH, | | 0.05 | | | 1.59 | |
| 6 months | | | | | | |
| container - close | | | | | | |
| 55° C./75% RH, | | 0.04 | | 0.09 | 1.27 | 1.68 |
| 1 week | | | | | | |
| container - close | | | | | | |
| 55° C./75% RH, | | 0.12* | | 0.5* | 1.06* | 1.17* |
| 1 week | | | | | | |
| container - close | | | | | | |
| 40° C./75% RH, | | 0.08* | | 0.5* | 1.5* | 0.91* |
| 6 months | | | | | | |
| container - close | | | | | | |

*packed in 150 cc HDPE bottle with 3 silica canisters of 1 g each.

1. A pharmaceutical formulation comprising:
(i) pellets comprising dipyridamole, and
(ii) pellets comprising acetylsalicylic acid,
wherein components (i) and (ii) are physically separated.

2. The pharmaceutical formulation of claim 1, wherein at least one of components (i) and (ii) is encapsulated in an capsule.

3. The pharmaceutical formulation of claim 1, wherein the pellets comprising acetylsalicylic acid are encapsulated in an inner capsule.

4. The pharmaceutical formulation of claim 2, wherein the pellets comprising dipyridamole are encapsulated in an outer capsule.

5. The pharmaceutical formulation of claim 4, wherein the inner capsule is encapsulated in the outer capsule.

6. The pharmaceutical formulation of claim 1, wherein each of the pellets comprising dipyridamole further comprises at least one pharmaceutically acceptable excipient.

7. The pharmaceutical formulation of claim 1, wherein each of the pellets comprising dipyridamole is coated with an extended release layer.

8. The pharmaceutical formulation of claim 7, wherein the extended release layer comprises an extended release polymer.

9. The pharmaceutical formulation of claim 8, wherein the extended release polymer is selected from the group consisting of ethylcellulose polymers, cellulose acetate polymers, hydroxypropyl methylcellulose polymers, polyvinylpyrrolidine polymers, methacrylic acid-ethyl acrylate copolymers, methacrylic acid-methyl methacrylate copolymers, and combinations thereof.

10. The pharmaceutical formulation of claim 8, wherein the extended release polymer is selected from hydroxypropyl methylcellulose polymers.

11. The pharmaceutical formulation of claim 7, wherein the extended release layer further comprises at least one pharmaceutically acceptable excipient.

12. The pharmaceutical formulation of claim 11, wherein the at least one pharmaceutically acceptable excipient in the extended release layer is a plasticizer.

13. The pharmaceutical formulation of claim 12, wherein the plasticizer is selected from the group consisting of acetyltributyl citrate, acetyltriethyl citrate, castor oil, diacetylated monoglycerides, dibutyl sebacate, diethyl phthalate, glycerin, polyethylene glycol, triacetin, tributyl citrate, triethyl citrate, and combinations thereof.

14. The pharmaceutical formulation of claim 13, wherein the plasticizer is triethyl citrate.

15. The pharmaceutical formulation of claim 1, wherein each of the pellets comprising dipyridamole further comprises at least one organic acid.

16. The pharmaceutical formulation of claim 15, wherein the organic acid has a pKa of about 5 or less than 5.

17. The pharmaceutical formulation of claim 16, wherein the organic acid has a pKa of about 4.5 or less than 4.5.

18. The pharmaceutical formulation of claim 17, wherein the organic acid has a pKa of about 4.2 or less than 4.2.

19. The pharmaceutical formulation of claim 15, wherein the organic acid is selected from the group consisting of fumaric acid, tartaric acid, citric acid, succinic acid, adipic acid, malic acid, and mixtures thereof.

20. The pharmaceutical formulation of claim 19, wherein the organic acid is tartaric acid.

21. The pharmaceutical formulation of claim 15, wherein each of the pellets comprising dipyridamole comprises
(a) an inner core comprising the at least one organic acid, and
(c) a drug layer comprising dipyridamole.

22. The pharmaceutical formulation of claim 21, wherein the inner core comprises a granulate comprising the at least one organic acid and at least one pharmaceutically acceptable excipient.

23. The pharmaceutical formulation of claim 21, wherein the drug layer further comprises at least one pharmaceutically acceptable excipient.

24. The pharmaceutical formulation of claim 21, wherein the drug layer comprises between about 10% and 100% by weight dipyridamole and between about 90% and 0% by weight at least one pharmaceutically acceptable excipient.

25. The pharmaceutical formulation of claim 24, wherein the drug layer comprises between about 30% and about 95% by weight dipyridamole and between about 70% and about 5% by weight at least one pharmaceutically acceptable excipient.

26. The pharmaceutical formulation of claim 25, wherein the drug layer comprises between about 50% and about 90% by weight dipyridamole and between about 50% and about 10% by weight at least one pharmaceutically acceptable excipient.

27. The pharmaceutical formulation of claim 26, wherein the drug layer comprises between about 70% and about 85% by weight dipyridamole and between 30% and about 15% by weight at least one pharmaceutically accepted excipient.

28. The pharmaceutical formulation of claim 21, wherein the inner core comprises between about 10% and 100% by weight the at least one organic acid and between about 90% and 0% by weight at least one pharmaceutically accepted excipient.

29. The pharmaceutical formulation of claim 28, wherein the inner core comprises between about 20% and about 90% by weight the at least one organic acid and between about 80% and about 10% by weight the at least one pharmaceutically accepted excipient.

30. The pharmaceutical formulation of claim 29, wherein the inner core comprises between about 30% to about 85% by weight the at least one organic acid and between about 70% to about 15% by weight the at least one pharmaceutically accepted excipient.

31. The pharmaceutical formulation of claim 30, wherein the inner core comprises between about 40% to about 80% by weight the at least one organic acid and between about 60% to about 20% by weight the at least one pharmaceutically acceptable excipient.

32. The pharmaceutical formulation of claim 21, wherein the inner core is coated with (b) an enteric coating layer, and wherein the drug layer is applied on top of the enteric coating layer.

33. The pharmaceutical formulation of claim 32, wherein the enteric coating layer comprises an enteric polymer.

34. The pharmaceutical formulation of claim 32, wherein the enteric polymer is selected from the group consisting of methacrylic polymers, methacrylic acid-ethyl acrylate copolymers, methacrylic acid-methyl methacrylate copolymers, and combinations thereof.

35. The pharmaceutical formulation of claim 32, wherein the enteric polymer is selected from the group consisting of hydroxypropyl methylcellulose phthalate, cellulose acetate phthalate, ethylcellulose phthalate, hydroxypropylmethylcellulose succinate, cellulose acetate succinate, hydroxypropylmethylcellulose hexahydrophthalate, cellulose acetate hexahydrophthalate, hydroxypropylmethylcellulose trimellitate, and combinations thereof.

36. The pharmaceutical formulation of claim 32, wherein the enteric coating layer further comprises at least one pharmaceutically acceptable excipient.

37. The pharmaceutical formulation of claim 36, wherein the pharmaceutically acceptable excipient in the enteric coating layer is a plasticizer.

38. The pharmaceutical formulation of claim 37, wherein the plasticizer in the enteric coating layer is selected from the group consisting of acetyltributyl citrate, acetyltriethyl citrate, castor oil, diacetylated monoglycerides, dibutyl sebacate, diethyl phthalate, glycerin, polyethylene glycol, triacetin, tributyl citrate, triethyl citrate, and combinations thereof.

39. The pharmaceutical formulation of claim 38, wherein the plasticizer in the enteric coating layer is triethyl citrate.

40. The pharmaceutical formulation of claim 7, wherein the extended release layer is applied on top of the drug layer.

41. The pharmaceutical formulation of claim 1, wherein each of the pellets comprising acetylsalicylic acid further comprises at least one pharmaceutically acceptable excipient.

42. The pharmaceutical formulation of claim 41, wherein the pellets comprising acetylsalicylic acid comprise between about 20% and about 70% by weight acetylsalicylic acid and between about 80% and about 30% by weight the at least one pharmaceutically acceptable excipient.

43. The pharmaceutical formulation of claim 42, wherein the pellets comprising acetylsalicylic acid comprise between about 30% and about 50% by weight acetylsalicylic acid and between about 70% and about 50% by weight the at least one pharmaceutically acceptable excipient.

44. The pharmaceutical formulation of claim 43, wherein the pellets comprising acetylsalicylic acid comprise between about 35% and about 45% by weight acetylsalicylic and between about 65% and about 55% by weight the at least one pharmaceutically acceptable excipient.

45. The pharmaceutical formulation of claim 6, comprising at least one pharmaceutically acceptable excipient selected from the group consisting of binders, diluent, disintegrants, lubricants, fillers, and mixtures thereof.

46. The pharmaceutical formulation of claim 1, wherein the particle size of the pellets comprising dipyridamole is between 1800 μm and 800 μm.

47. The pharmaceutical formulation of claim 46, wherein the particle size of the pellets comprising dipyridamole is between 1400 μm and 1000 μm.

48. The pharmaceutical formulation of claim 1, wherein the particle size of the pellets comprising acetylsalicylic acid is between 1600 μm and 400 μm.

49. The pharmaceutical formulation of claim 46, wherein the particle size of the pellets comprising acetylsalicylic acid is between 1000 μm and 500 μm.

50. The pharmaceutical formulation of claim 5, comprising an outer capsule that encapsulates:
(i) the pellets comprising dipyridamole, wherein each of the pellets comprise:
(a) an inner core comprising a granulate comprising at least one organic acid,
(b) an enteric coating on top of the inner core,
(c) a drug layer comprising dipyridamole on top of the enteric coating, and
(d) an extended release layer; and
(ii) an inner capsule that encapsulates the pellets comprising acetylsalicylic acid.

51. The pharmaceutical formulation of claim 1, wherein the weight of any one of dipyridamole's degradants in the formulation is not more than 0.2% by weight of the initial weight of the dipyridamole in the formulation, after storage under one of the following conditions:
a) at 25° C. and 60% relative humidity for 2 years,
b) at 30° C. and 60% relative humidity for 6 months, and
c) at 40° C. and 75% relative humidity for 3 months.

52. The pharmaceutical formulation of claim 1, wherein the weight of any one of acetylsalicylic acid's degradants in the formulation is not more than 3% by weight of the initial weight of the acetylsalicylic acid in the formulation, after storage under one of the following conditions:
a) at 25° C. and 60% relative humidity for 2 years,
b) at 30° C. and 60% relative humidity for 6 months, and
c) at 40° C. and 75% relative humidity for 3 months.

53. A process for producing the pharmaceutical formulation of claim 5, comprising:
(1) placing the pellets comprising acetylsalicylic acid in the inner capsule,
(2) placing the inner capsule in the outer capsule, and
(3) placing the pellets comprising dipyridamole in the outer capsule.

54. (canceled)

55. A method of reducing the risk of stroke in a patient, comprising administering the pharmaceutical formulation of claim 1 to a patient who has had a stroke or a transient ischemic attack.

56. The pharmaceutical formulation of claim 1 which can be administered to a patient as sprinkled pellets.

57. A method of administering the pharmaceutical formulation of claim 1, comprising (a) sprinkling the pellets comprising dipyridamole and the pellets comprising acetylsalicylic acid according to claim 1 on food, and (b) administering the food orally.

58. A plurality of pellets, comprising between about 20% and about 70% by weight acetylsalicylic acid and between about 80% and about 30% by weight at least one pharmaceutically acceptable excipient, wherein the particle size of the pellets is between about 1000 μm and about 200 μm.

59-65. (canceled)
